(19) United States
(12) Patent Application Publication
(10) Pub. No.: US 2017/0014339 A1
(43) Pub. Date: Jan. 19, 2017

(54) Tear Substitute, Fluid for Being Used as a Tear Substitute, and Method for Producing a Tear Substitute

(71) Applicant: i.com medical GmbH, Munich (DE)

(72) Inventor: Wolfgang G. K. MUELLER-LIERHEIM, Munich (DE)

(21) Appl. No.: 14/802,460

(22) Filed: Jul. 17, 2015

(51) Int. Cl. A61K 9/00; A61K 31/728

(52) U.S. Cl. CPC A61K 9/0048; A61K 31/728

(57) ABSTRACT

A tear substitute having hyaluronan has a viscoelastic flow characteristics with the zero shear viscosity being ≧50 mPa·s, and the shear viscosity at 1000 s⁻¹ being ≦12 mPa·s or having hyaluronan with an intrinsic viscosity [η]>2.5 m³/kg and a concentration of <0.2% w/v. A fluid with the aforementioned specifications is used as a tear substitute. A method of producing a tear substitute comprises the use of substances for reaching a viscoelastic flow characteristics with the zero shear viscosity being 50 mPa·s, and the shear viscosity at 1000 s⁻¹ being ≦12 mPa·s. Alternatively the inventive method comprises the use of hyaluronan with an intrinsic viscosity [η]>2.5 m³/kg and a concentration of <0.2% w/v.

### FIELD OF THE INVENTION

The present invention concerns a tear substitute, usually also called eye drops, for the treatment of ocular surface disease like dry eye disease, a fluid to be used as a tear substitute, as well as a method for producing a tear substitute.

### BACKGROUND OF THE INVENTION

Tear substitutes play an essential role in the treatment of ocular surface disease. As a lubricant they are intended to minimize the friction between lid and corneal epithelium during blinking Their water binding capacity contributes to the hydration of irritated eyes.

For the physiological function of natural human tears it is characteristic that they exhibit high viscosity in the absence of shear stress as typically 65 mPa·s in the open eye, and low viscosity during blinking as typically 10 mPa·s. This ensures high stability of the tear film in the open eye and low shear stress on the corneal epithelium during blinking However, the flow characteristics of most of the currently available tear substitutes does not mimic the rheology of human tears. Most eye drops exhibit an almost Newtonian flow characteristics, i.e. they have either low or high viscosity both in the open eye and during blinking

Over the last 15 years numerous hyaluronan eye drops have become available, claiming viscoelastic flow characteristics and long persistence in the eye. Measurements of the flow characteristics of more than 40 different hyaluronan (HA) eye drop brands resulted in that those containing low HA concentration act like Newtonian fluids with low viscosity whereas those with high HA concentration (gels) have high viscosity during blinking causing blurring and excess shear stress on the corneal epithelium, and nevertheless have lower viscosity than natural tears in healthy eyes between blinking

Only the use of very high molecular weight HA (≈3 MDa) in approximately 0.15 percent concentration can mimic the flow characteristics of human tears. The relation between molecular weight and intrinsic viscosity [η] in m³/kg is given through the Mark-Houwink equation

[η]=k·(Mᵣₘ)^{a}

with Mᵣₘ being the molecular mass in MDa

and the coefficients

k=1.3327·10⁻⁴

and

a=0.6691

which values for k and a having been found as most predictive.

Another important aspect is the biochemical function of HA. HA is the most important component of the extracellular matrix of multilayer epithelia without blood supply, like the corneal epithelium. HA organizes the extracellular matrix, provides water storage and retention, is responsible for the diffusion of nutrients and metabolic products, controls keratinocyte proliferation and differentiation, is an efficient radical scavenger in cases of UV exposure, infection, oxidative stress and inflammatory processes with tissue necrosis, and facilitates the migration of epithelial cells during wound healing. The molecular weight of HA in the extracellular matrix of healthy epithelia is about 3 to 4 MDa. In case of increased HA degradation, e.g. due to inflammatory processes, HA exhibits a molecular weight dependent signal function. While high molecular weight HA acts anti-angiogenetically and immunosuppressively, small to medium size HA molecules induce inflammatory factors and stimulate immunity and angiogenesis. The binding of free HA to cell membrane receptors protects the cells from lymphocytic and macrophagic attack.

The half-life of HA in the epithelial extracellular matrix is only about 24 hours. Due to intrinsic aging, commencing from the fifth decade of life the amount of freely available and extractible HA in the intercellular spaces decreases rapidly. This is the main reason for the loss of hydration and thickness of the epidermis in older people, suggesting that HA may also play an essential role in age-related dry eye.

US 2004/0013729 A1 discloses to use methylcellulose for increasing the time available for agents to contact the cornea. Although such methylcellulose in US 2004/0013729 A1 is named a viscoelastic polymer, this is against the true meaning of viscoelasticity because methylcellulose does not have viscoelastic behavior but largely acts like a Newtonian fluid.

### SUMMARY OF THE INVENTION

The objective of the invention is to provide for tear substitute for improved treatment of ocular surface disease like dry eye disease.

This objective is reached with tear substitute according to claim 1. Further advantageous and preferred embodiments are given in the dependent claims and through combinations thereof.

According to the present invention, a tear substitute comprises hyaluronan is characterized by a viscoelastic flow characteristics with the zero shear viscosity being ≧50 mPa·s, and the shear viscosity at 1000 s⁻¹ being ≦12 mPa·s. The tear substitute comprises hyaluronan with a hyaluronan intrinsic viscosity [η]>2.5 m³/kg and a concentration of <0.2% w/v. It is also contemplated by the present invention that the tear substitute has the hyaluronan intrinsic viscosity [η]≧2.9 m³/kg.

According to the invention, the tear substitute further comprises substances naturally present in a human eye; and substances which are pharmacologically, metabolically, immunologically and/or anti-microbial effective, or being preservative-free. The tear substitute further comprises glycosaminoglycanes, electrolytes, and buffers. The tear substitute further provides that the glycosaminoglycanes comprise hyaluronan, and/or the electrolytes comprise sodium chloride, and/or the buffers comprise a phosphate buffer in concentration ≦1.45 mmol/l or trometamol.

According to the invention, the tear substitute comprises ≦2% w/v of substances which are pharmacologically, metabolically, immunologically and/or anti-microbial effective. A further embodiment of the invention provides that the tear substitute comprises ≦0.1% w/v of substances which are pharmacologically, metabolically, immunologically and/or anti-microbial effective. A further embodiment of the invention provides that the tear substitute comprises ≦0.01% w/v substances which are pharmacologically, metabolically, immunologically and/or anti-microbial effective.

Further according to the invention, a fluid comprises hyaluronan and having a viscoelastic flow characteristics with the zero shear viscosity being ≧50 mPa·s, and the shear viscosity at 1000 s⁻¹ being ≦12 mPa·s, characterized in that said fluid being used as a tear substitute. A fluid comprises hyaluronan with an intrinsic hyaluronan viscosity [η]>2.5 m³/kg and a concentration of <0.2% w/v, characterized in that said fluid being used as a tear substitute. It is also contemplated by the present invention that the fluid has the hyaluronan intrinsic viscosity [η]≧2.9 m³/kg.

According to the invention, the fluid further comprises substances naturally present in a human eye; and substances which are pharmacologically, metabolically, immunologically and/or anti-microbial effective, or being preservative-free. The fluid further comprises glycosaminoglycanes, electrolytes, and buffers. The fluid further comprises the glycosaminoglycanes comprise hyaluronan, and/or the electrolytes comprise sodium chloride, and/or the buffers comprise a phosphate buffer in concentration ≦1.45 mmol/l or trometamol.

According to the invention, the fluid comprises 2% w/v of substances which are pharmacologically, metabolically, immunologically and/or anti-microbial effective. A further embodiment of the invention provides that the fluid comprises 0.1% w/v substances which are pharmacologically, metabolically, immunologically and/or anti-microbial effective. A further embodiment of the invention provides that the fluid compises 0.01% w/v substances which are pharmacologically, metabolically, immunologically and/or anti-microbial effective.

According to the invention, a method of producing a tear substitute comprises a step of using viscosity-increasing substances for reaching a viscoelastic flow characteristics with the zero shear viscosity being ≧50 mPa·s, and the shear viscosity at 1000 s⁻¹ being ≦12 mPa·s. A method of producing a tear substitute comprises a step of using hyaluronan with a hyaluronan intrinsic viscosity [η]>2.5 m³/kg and a concentration of <0.2% w/v. The method also provides that the hyaluronan intrinsic viscosity [η]≧2.9 m³/kg.

According to the method, the viscosity-increasing substances are added towards or during or as a final step. A step of mixing is carried out so as to reach or until reaching a homogeneous mixture. The method also contemplates a step of providing as a basis initially purified water or water for injection. The method also contemplates first adding electrolytes, buffers and substances which are not increasing the viscosity to the purified water or water for injection.

### DETAILED DESCRIPTION OF THE PREFERRED EMBODIMENTS

In the following the invention is explained by way of examples only.

According to a first aspect of the invention there is provided tear substitute containing hyaluronan and having a viscoelastic flow characteristics with the zero shear viscosity being ≧50 mPa·s, and the shear viscosity at 1000 s⁻¹ being ≦12 mPa·s.

Viscoelasticity is defined as characteristics of a fluid having both viscous and elastic properties. The zero shear viscosity is determined as the steady shear plateau viscosity at vanishing shear rate. For highly viscous formulations, measurement with a controlled stress rheometer is preferred which is well known to a man skilled in the art.

The aforementioned first aspect also is fulfilled by a fluid, which contains hyaluronan and has a viscoelastic flow characteristics with the zero shear viscosity being ≧50 mPa·s, and the shear viscosity at 1000 s⁻¹ being ≦12 mPa·s, wherein said fluid being used as tear substitute.

Both the tear substitute and the fluid of the first aspect preferably being at least essentially mucin-free or in other words having a mucin concentration of <0.3% w/v. This means that the flow behaviour or properties essentially is reached or adjusted by hyaluronan and not by mucin naturally present in the tear fluid and mainly responsible for the flow behavior thereof.

Furthermore, within the scope of said first aspect of the present invention there also is a method of producing a tear substitute, comprising the use of substances for reaching a viscoelastic flow characteristics with the zero shear viscosity being ≧50 mPa·s, and the shear viscosity at 1000 s⁻¹ being ≦12 mPa·s. Mucin concentration if any is less than 0.3% w/v.

Next, according to a second aspect of the invention there is provided a tear substitute containing hyaluronan with an intrinsic viscosity [η]>2.5 m³/kg and a concentration of <0.2% w/v. Preferably the hyaluronan intrinsic viscosity [η]≧2.9 m³/kg.

The aforementioned second aspect also is fulfilled by a fluid, containing hyaluronan with an intrinsic viscosity [η]>2.5 m³/kg and a concentration of <0.2% w/v, wherein said fluid being used as a tear substitute. Preferably the hyaluronan intrinsic viscosity [η]≧2.9 m³/kg.

Both the tear substitute and the fluid of the second aspect preferably being at least essentially mucin-free or in other words having a mucin concentration of <0.3% w/v. This means that the flow behaviour or properties essentially is reached or adjusted by hyaluronan and not by mucin naturally present in the tear fluid and mainly responsible for the flow behavior thereof.

Also, within the scope of said second aspect of the present invention there is a method of producing a tear substitute, comprising the use of hyaluronan with an intrinsic viscosity [η]>2.5 m³/kg and a concentration of <0.2% w/v. Mucin concentration if any is less than 0.3% w/v. Preferably the hyaluronan intrinsic viscosity [η]≧2.9 m³/kg.

Preferably there are contained or used only substances which are naturally present in the human eye plus eventually substances which are pharmacologically, metabolically, immunologically and/or anti-microbial effective.

It is furthermore preferred that are contained or used glycosaminoglycanes, electrolytes, and buffers. Especially the glycosaminoglycanes include hyaluronan, and/or the electrolytes include sodium chloride, and/or the buffers include a phosphate buffer in concentration ≦1.45 mmol/l or trometamol.

An even further preferred embodiment is the content or use of ≦2% w/v, especially ≦0.1% w/v, and especially preferred ≦0.01% w/v substances which are pharmacologically, metabolically, immunologically and/or anti-microbial effective.

With regard to the method within the scope of the present invention it is further preferred, that substances which are increasing the viscosity are added towards or during or as a final step.

According to another preferred embodiment of the method of the present invention, mixing is carried out so as to reach or until reaching a homogeneous mixture. As an alternative or in addition it is preferred to initially provide for purified water or water for injection as a basis, and then, especially, electrolytes, buffers and substances which are not increasing the viscosity are added at first to the purified water or water for injection.

For comparison with tear substitutes or eye drops having other specifications tests of the tear substitute having the following specifications were made with patients with ocular surface disease and achieved excellent and the best results:

| | | |
|---|---|---|
| | | Test |
| Characteristic | Specification | Method |

| | | |
|---|---|---|
| appearance | clear and colorless solution, | Ph. Eur. |
| | free from visible impurities | |
| pH value | 6.8-7.6 | Ph. Eur. |

| | | | |
|---|---|---|---|
| osmolality | 240-330 | mosmol/kg | Ph. Eur. |
| HA concentration | 0.10-0.19% | w/v | Ph. Eur. |
| NaCl concentration | 7.6-10.5 | g/l | Ph. Eur. |

| | | |
|---|---|---|
| sterility | sterile | Ph. Eur. |

| | | | |
|---|---|---|---|
| Phosphate concentration | 1.0-1.4 | mmol/l | Ph. Eur. |

The invention is described only exemplarily by the embodiments in the description and drawings and is not limited thereto but rather includes all variations, modifications, substitutions, and combinations the expert may take from the complete documents of this application under consideration of and/or combination with his specific knowledge.

What is claimed is:

1. A tear substitute comprising hyaluronan and having a viscoelastic flow characteristics with
the zero shear viscosity being ≧50 mPa·s, and
the shear viscosity at 1000 s⁻¹ being ≦12 mPa·s.

2. A tear substitute comprising hyaluronan with a hyaluronan intrinsic viscosity [η]>2.5 m³/kg and a concentration of <0.2% w/v.

3. The tear substitute according to claim 2, wherein the hyaluronan intrinsic viscosity [η]2.9 m³/kg.

4. The tear substitute according to claim 1, further comprising:
substances naturally present in a human eye; and
substances which are pharmacologically, metabolically, immunologically and/or anti-microbial effective, or being preservative-free.

5. The tear substitute according to claim 1, further comprising glycosaminoglycanes, electrolytes, and buffers.

6. The tear substitute according to claim 5, wherein the glycosaminoglycanes comprise hyaluronan, and/or the electrolytes comprise sodium chloride, and/or the buffers comprise a phosphate buffer in concentration 1.45 mmol/l or trometamol.

7. The tear substitute according to claim 1, comprising ≦2% w/v of substances which are pharmacologically, metabolically, immunologically and/or anti-microbial effective.

8. The tear substitute according to claim 7, comprising ≦0.1% w/v of substances which are pharmacologically, metabolically, immunologically and/or anti-microbial effective.

9. The tear substitute according to claim 8, comprising ≦0.01% w/v substances which are pharmacologically, metabolically, immunologically and/or anti-microbial effective.

10. A fluid comprising hyaluronan and having a viscoelastic flow characteristics with
the zero shear viscosity being ≧50 mPa·s, and
the shear viscosity at 1000 s⁻¹ being ≦12 mPa·s, characterized in that said fluid being used as a tear substitute.

11. A fluid comprising hyaluronan with an intrinsic hyaluronan viscosity [η]>2.5 m³/kg and a concentration of <0.2% w/v, characterized in that said fluid being used as a tear substitute.

12. The fluid according to claim 11, wherein the hyaluronan intrinsic viscosity [η]≧2.9 m³/kg.

13. The fluid according to claim 10, further comprising:
substances naturally present in a human eye; and
substances which are pharmacologically, metabolically, immunologically and/or anti-microbial effective, or being preservative-free.

14. The fluid according to any of claims 10, further comprising glycosaminoglycanes, electrolytes, and buffers.

15. The fluid according to claim 14, wherein the glycosaminoglycanes comprise hyaluronan, and/or the electrolytes comprise sodium chloride, and/or the buffers comprise a phosphate buffer in concentration ≦1.45 mmol/l or trometamol.

16. The fluid according to any of claims 10, comprising ≦2% w/v of substances which are pharmacologically, metabolically, immunologically and/or anti-microbial effective.

17. The fluid according to claim 16, comprising ≦0.1% w/v substances which are pharmacologically, metabolically, immunologically and/or anti-microbial effective.

18. The fluid according to claim 17, comprising ≦0.01% w/v substances which are pharmacologically, metabolically, immunologically and/or anti-microbial effective.

19. A method of producing a tear substitute, comprising a step of using viscosity-increasing substances for reaching a viscoelastic flow characteristics with
the zero shear viscosity being ≧50 mPa·s, and
the shear viscosity at 1000 s⁻¹ being ≦12 mPa·s.

20. A method of producing a tear substitute, comprising a step of using hyaluronan with a hyaluronan intrinsic viscosity [η]>2.5 m³/kg and a concentration of <0.2% w/v.

21. The method according to claim 20, wherein the hyaluronan intrinsic viscosity [η]≧2.9 m³/kg.

22. The method according to claim 19, wherein the viscosity-increasing substances are added towards or during or as a final step.

23. The method according to claim 19, comprising a step of mixing carried out so as to reach or until reaching a homogeneous mixture.

24. The method according to claim 19, comprising a step of providing as a basis initially purified water or water for injection.

25. The method according to claim 24, comprising first adding electrolytes, buffers and substances which are not increasing the viscosity to the purified water or water for injection.
